(19) United States
(12) Plant Patent Application Publication
(10) Pub. No.: US 2023/0276722 P1
(43) Pub. Date: Aug. 31, 2023

(54) Pests Repellant Fruit Vine

(50) ;

(71) Applicant: Miah Muhammad Adel, Pine Bluff, AR (US)

(72) Inventor: Miah Muhammad Adel, Pine Bluff, AR (US)

(21) Appl. No.: 17/942,119

(22) Filed: Sep. 10, 2022

(51) Int. Cl. A01H 6/34

(52) U.S. Cl. PLT/156

(57) ABSTRACT

Cucurbitaceae family snake gourd (Trichosanthes anguina L.), grown in many Asian countries; green, tender stems, forked tendrils, heart-shaped shaped or palmately broad leaves; long lacy fringed, five-petalled, day-night open, white unisexual flowers; multifarious health benefiting fruits; resistant to major fungal, bacterial, viral diseases, repellant to aphids, insects, and beetles (Ceroterma trifurcata) from pumpkin, squash, and bean plants; replaces bee-killing and water resources polluting pesticides.

Latin name of the plant claimed: Trichosanthes anguina L.

Variety: Trichosanthes cucumerina var. anguina (L.) Haines—cultivated variant Trichosanthes cucumerina var. cucumerina—wild variant

### BACKGROUND OF THE INVENTION

The primary objective was to avoid the use of environment polluting and bee population declining chemical pesticides like Sevin, Malathion, Diazinon, Neonicotinoids (Neonics) for gardens growing pumpkin, squash, and beans, and to avoid killing pests manually.

The present invention relates to a novel and distinct variety of Trichosanthes cucumerina that resists major fungal, bacterial, and viral diseases, and repels aphids, insects, and beetles (Cerotoma trifurcata) from pumpkin, squash, and bean plants if the discovered plant is uniformly interposed among the target fruit plants.

### BRIEF SUMMARY OF THE INVENTION

I am an avid gardener since my childhood days. Pumpkin, squash, beans, and snake gourds are my favorite fruits. In growing Pumpkin, squash, and bean, plant leaves are skeletonized by beetles, and beans are made holes and incubators of pests. Killing the pests manually was quite bothering since each leaf was to be checked individually as the plant grew. On the contrary, snake gourd leaves were free from pests. These were quite healthy growing. I thought of planting them among pumpkin, squash, and bean vines. As was expected, there was less infestation than the previous year. Next year, I planned to interpose snake gourd plants as uniformly as possible among the target plants. Pumpkin and squash beetles were gone. Bean leaves were unharmed. Beans were in good shape.

### BRIEF DESCRIPTION OF DRAWINGS

The accompanying illustrations show typical specimens of the vegetative growth, flowers, and variety in colors and sizes as true as reasonably possible.

FIG. 1. 1^{st} inset: Whitestripped greenskinned snake gourd; 2^{nd} inset: Whitestripped whiteskinned snake gourd; 3^{rd} inset: Picked snake gourd from trellises (top); whole snakegourd cut into pieces for cooking (bottom); 4^{th} inset: Mature, ripe yellow colored snake gourd (top) (https://www.dreamstime.com/snake-gourd-trichosanthes-cucumerina-serpent-gourd-chichinda-padwal-die-schlangenhaargurke-schlangengurke-chinesische-gurke-snake-image162461541); seeds taken out from inside of a ripe snake gourd (bottom) (https://www.krsnastreats.org/recipes/entrees/item/snake-gourd-pasta-sauce-no-tomatoes).

FIG. 2. 1^{st} inset: A dicotyledonous baby plant seedlings with two embryonic leaves in about two weeks days after germination; 2^{nd} inset: Baby plant without tendrills; 3^{rd} inset: Vigorously growing plant with tendrills climbing a vertical trellis; 4^{th} and 5^{th} insets: Vertically rising vigorously growing snake gourd plant with tendrills.

FIG. 3. 1^{st} inset: heart-shaped leaf; 2^{nd} inset: 3-lobed leaf; 3^{rd} inset: 6-lobed leaf; 4^{th} inset: 7-lobed leaf, five-petalled white unisexual flowers with long lacy fringes; 5^{th} inset: Vigorously growing plants climbing a vertical trellis with forked tendrill

FIG. 4. 1^{st} inset: Beetles made holes in hyacinth bean plant leaves; 2^{nd} inset: Spotless heart-shaped snake gourd leaves and the three-piece heart shaped hyacinth flat bean leaves; 3^{rd} inset: Heart-shaped leaf, forked tendrils, and baby snake gourd of white variety, female flower with thick stem; 4^{th} inset: No pumpkin beetles to lay eggs on the under side of leaves; 5^{th} inset: male flowers borne on short stalks of about equal length at equal distances along an elongated axis (peduncle length 10 to 30 cm) and open in succession toward the apex.

FIG. 5. 1^{st} inset: Wilting of the plant under drought and temperature about 100° F. or above; 2^{nd} inset through 4^{th} insets: successive stages of blight attacked leaves.

### DETAILED BOTANICAL DESCRIPTION

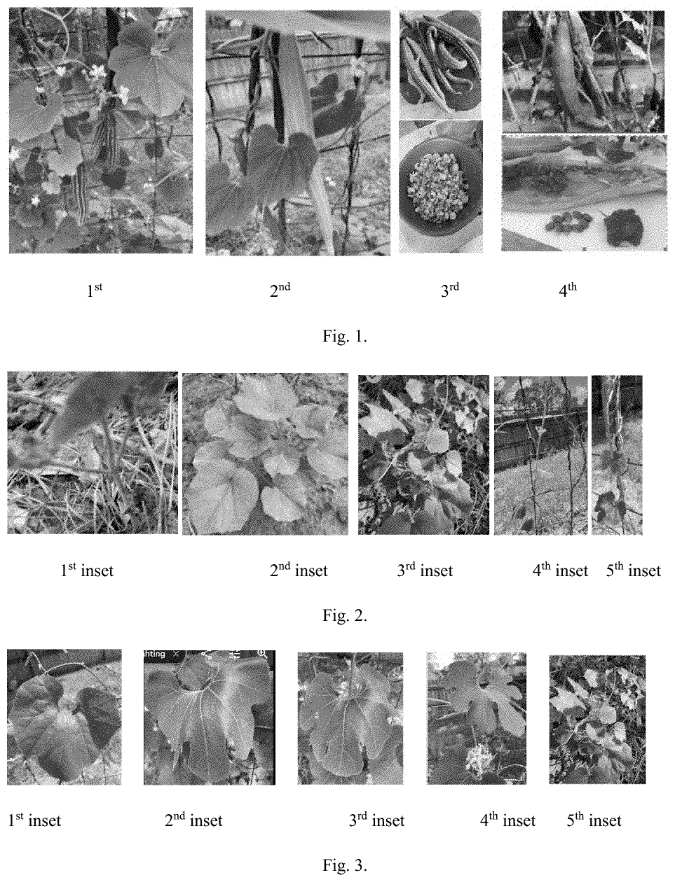

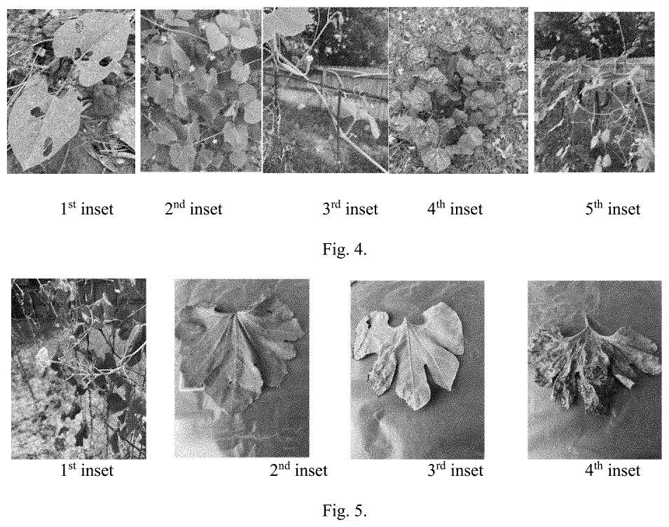

The plant's phenotype is determined by both their genomic makeup (genotype) and environmental factors with variations in environmental, climatic, and cultural conditions, as it has not been tested under all possible environmental conditions.
Snake gourd: (Trichosanthes cucumerina), also known as serpent gourd, rapid-growing fruit vine belonging to the gourd family (Cucurbitaceae);
Scientific classification: Kingdom: Plantae; Division: Magnoliophyta; Class: Mangnoliopsida; Order: Curcubitales; Family: Curcubitaceae; Genus: Trichosanthes; Species: Cucumerina;
Regional names: Trichosanthes cucumerina is known as Chicinga in Bengali, Chichinda or Padwal in Hindi, Potlakaaya in Telugu, Pudalankaai in Tamil and Padavalanga in Malayalam, potlakaaya in Telugu, pudalankaai in Tamil, aduvalakaay in India's Karnataks state, padavalanga, in Malayalam, Galartori in Punjabi, padavali in Gujarathi, Chachinda in Hindi (3,4); as serpent vegetal in France, Schlangengurke in Germany, Karasu-uri-zoku in Japan, Patola in Srilanka, Zucchetta cinese in Italy, Abóbora-serpente in Portugal, Käärmekurkku in Finland, Buap nguu Ma noi in Thailand, Yilan kabagi in Turkey, Calabaza anguina in Spain (Devi, 2017)
Taxonomy: The plant's family, predominantly distributed in the tropics, commonly known as melons, gourds or cucurbits that includes cucumbers, squashes (including pumpkins), luffas, melons (including watermelons); one of the most genetically diverse groups of food plant in the plant kingdom; Trichosanthes (100 species), Cayaponia (60 species), Momordica (47 species), Gurania (40 species), Sicyos (40 species) and Cucumis (34 species) major major genera under this family (Devi, 2017).
Biosystematics: Two cultivated species Trichosanthes anguina L. and Trichosanthes dioica Roxb.; Important wild species Trichosanthes bracteata (Lam.) Voigt. (Syn. Trichosanthes palmata Roxb.), largely variable Trichosanthes cucumerina L., Trichosanthes lobata, Trichosanthes wallichiana (syn. Trichosanthes multiloba Clarke), Trichosanthes nervifolia L., Trichosanthes cordata Roxb., Trichosanthes japonica and Trichosanthes shikokiana; botanical name Trichosanthes cucumerina L. , most common is Trichosanthes anguina L. (Devi, 2017).
Cytology: Trichosanthes chromosomes, n=11 and 2n=22; Trichosanthes bracteate and Trichosanthes cucumeroides n=22 and 2n=44. Tetraploids (2n=44) and hexaploids (2n=66) in Trichosanthes palmate. Induced polyploids in Trichosanthes anguina quadrivalents, trivalents, bivalents and univalent; Only Trichosanthes anguina and Trichosanthes cucumerina monoecious, all others dioecious. Species with 2n−=22 chromosomes metacentric to submetacentric medium-sized (5.74 mm to 1.48 mm) chromosomes; only three pairs of chromosomes with secondary constriction in Trichosanthes anguina and Trichosanthes cucumerina; XY sex chromosomes in Trichosanthes cucumeriodes and Trichosanthes japonica at meiosis and in Trichosanthes multiloba at metaphase. Trichosanthes anguina, Trichosanthes cucumerina and Trichosanthes lobata crossable among themselves with fertile hybrids indicating their close relationship (Devi, 2017).
Seeds: Imbedded in soft red pulp in ripe snake gourd, half-ellipsoid, somewhat compressed, undulate, hard, corrugated, about 1 cm long, about ¾ cm across, greyish-brown, black, sculptured, undulate(4^{th} inset bottom in FIG. 1).
Seedlings: Seeds planting about 1 m apart at depth about 2.5 cm in well-drained, humus-rich adequate moisture content soil with Ph 6.5-7.0 in sunny locations with at least 6 hrs direct sunlight; tiny and few or no fruits for lesser amount of sunlight; dicotyledonous seedlings with two embryonic leaves (1^{st} inset in FIG. 2) in about two weeks in temperature between 80° F. and 95° F. (27° C. to 35° C.); longer timing of weeks for seedlings in lower temperature. For temperatures nearing 100° F. and above, leaves get wilted in the scorching sun. Blight attacks under drought and such high temperatures (FIG. 5).
Plant: Monoecious annual, branchy, slender, green, disagreeable odor, stems up to 5 or 6 meters long, diameter of 5 mm, possibly largest number of fruits producer among all Cucurbitaceae; vigorous growth; climb up vertical supports using tendrils and twining stems to sprawl over its top or the trellises (3^{rd} through 5^{th} insets in FIG. 2, 5^{th} inset in FIGS. 3, 2^{nd} and 3^{rd} insets in FIG. 4); plant very variable in the shape of the fruit, some named varieties, traditional cultivars 6-10 fruits per year, some improved cultivars more fruits per year; garden grown, vegetable, medicinal, and ornamental plant; drought intolerant. Manual weed control required. Watering required to keep the soil humid, no flooding.
Leaves: Base broadly heart-shaped or palmately broad 3, 5, 6, or 7-lobed leaves rounded or obtuse appear, rounded outline, 8 to 12 cm long and broad, sinuses broad or narrow and rounded (1^{st} through 4^{th} insets in FIG. 3). Greenish nature of leaves fades away for shortfalls of nitrogen.
Tendrils: Forked tendrils appearance before the characteristic odor at about 1 m high (3^{rd} through 5^{th} insets in FIG. 2, 5^{th} inset in FIG. 3, 3^{rd} inset in FIG. 4).
Manure: Periodic application of composted vegetable matter and 13-13-13.
Flowers: Flowering by 1.5 m high, five-petalled white unisexual flowers with long lacy fringes (4th inset in FIG. 3); small, white flowers in bloom day and night; male and female flowers separate; male flowers borne on short stalks of about equal length at equal distances along an elongated axis (peduncle length 10 to 30 cm) and open in succession toward the apex (5^{th} inset in FIG. 4); female ones apart by the long lump on the stem; solitary, sessile, single celled ovary, long and with hairy stigmas (3^{rd} inset in FIG. 4).
Pollination: Insects pollinated; manual pollination by snapping off the male flowers and gently rubbing them on the female ones to transfer the pollen; female flower wilting after successful pollination; lump growth on the stem begins to grow; appearances of baby snake gourds; rapid growth (3^{rd} inset in FIG. 4).
Fruit: Oddly shaped fruits—very slender, long, cylindrical berry, often twisted, green or white when immature, pickable at about 30 cm; immature fruits harvestable about two months after sowing seeds; cropping continues for up to four months or more depending on the weather; delayed picking hardened fruit prior to turning yellowish red (4^{th} insets in FIG. 1); 10 fruits or more per plant; 30 cm mature fruit 5-8 cm middle diameter, thickness, growth dependent weights.
Edible: Young whole fruits cut into pieces for cooking (3^{rd} inset (bottom) in FIG. 1); mature fruits' red, soft sweet testing pulp eaten as a tomato substitute in African countries (4^{th} inset in FIG. 1); bright red pulp around mature seeds extracted for tomato-like cooking.
Pests: The described snake gourds herein resistant to powdery mildew, and repellent to leaf beetles, aphids, snails, slugs, and caterpillars.
Snake gourd nutritional content: The snake gourd nutrition facts for a 100 g serving are as follows: Calories 86.2 kcal,
Macronutrients: Total Fat 3.9 g, Saturated Fat 0.5 g, Total carbohydrate 12.5 g, Dietary Fiber 0.6 g, Protein 2.0 g, Cholesterol 0.0 mg, Sodium 33.0 mg, Potassium 359.1 mg, Vitamins: Vitamin A 9.8 % Vitamin B6 11.3%,. Vitamin C 30.5%, Vitamin E 1.1%, Minerals: Calcium 5.1 %, Magnesium 6.7%, Phosphorus 5.0%, Zinc 7.2%, Iron 5.7%, Manganese 12.5%, and Iodine 5.9% (Bharat, 2022; Dube, 2022).
Snake gourd health benefits: Accelerates weight loss, augments heart function, detoxifies kidneys, enhances digestive system, strengthens respiratory processes, complements ketogenic diet, soothes depression and anxiety, resolves acid reflux, mitigates Polycystic ovary syndrome (PCOS); seed remedies hypertension, cures diarrhea, manages diabetes symptoms; snake gourd naturally moisturizes skin, combats skin infections, promotes hair growth, tackles excessive dandruff, snake gourd hair gel; in Ayurveda: snake gourd juice battles fevers, fights jaundice, remedies heart ailments, treats alopecia, recovers joint illnesses, uplifts immunity, regulates thyroid, alleviates insomnia (Bharat, 2022)
Pesticides substitute: Snake gourd effective repellant for pumpkin and squash bugs (ALMANAC), hyacinth bean bugs and aphids (Plant Village); replacement for Sevin, Malathion, Diazinon, and Neonicotinoids (Neonics) pesticides used in growing pumpkins, squash, and beans and thus saves bee population, keeps water bodies and stream safe. So, environmental savior. Adverse environmental effects of pesticides—sevin, malathion, diazinon, and neonicotinoids (neonics)—particularly, the bee population and the water quality. Sevin and malathion efficacy in killing pumpkin beetles (Khan and Jehangir, 2000; Hasan et al., 2011). Recommended neonicitinoid class dinotefuran lambda cyhalothrin, esfenvelerate, and pyrethrin for squash bugs (UC Pest Management Guidelines) Pumpkin and squash bugs make holes in leaves, and Hyacinth (Dolichos Lablab Purpureus) bug makes holes in leaves and the beans. Also, aphids attack these plants. The suggested remedies are pyrethrum and Bacillus thuringiensis (Smart Gardener); and the references therein). Sevin, malathion, diazinon, and neonicotinoids (neonics) toxic for honeybees (Gill and Raine, 2014). Pesticides accumulation in streams and water bodies can enter aquatic organisms, risky for human consumption (Stockpoole, 2017).

### REFERENCES

1. Devi, N. D. 2017. Medicinal Values of Trichosanthus cucumerina L. (Snake Gourd)—A Review, British Journal of Pharmaceutical Research, 16(5): 1-10, 2017; Article no.BJPR.3357, ISSN: 2231-2919, NLM ID: 101631759

2. Bharat, D. 2022, Snake Gourd: Health Benefits, Nutrition, Uses For Skin And Hair, Recipes, Side Effects, available from https://www.netmeds.com/health-library/post/snake-gourd-health-benefits-nutrition-uses-for-skin-and-hair-recipes-side-effects

3. ALMANAC, Aug. 29, 2022, Squash Bugs: How to Get Rid of Squash Bugs | The Old Farmer's Almanac, available in (https://www.almanac.com/pest/squash-bugs

4. Plant Village, Bean, available in https://plantvillage.psu.edu/topics/bean/infos

5. Khan, S. M. and Jehangir, M. 2000. Efficacy of different concentrations of sevin dust against red pumpkin beetle (Aulacophora foveicollis (Lucas) causes damage to muskmelon Cucumis melo crop. Pakistan Journal of Biological Science., 3(1): 183-185.

6. Hasan, M. K., Uddin M. M., and Haque, M. A. 2011. EFFICACY OF MALATHION FOR CONTROLLING RED PUMPKIN BEETLE, Aulacophora foveicollis (LUCAS) IN CUCURBITACEOUS VEGETABLES, Progress. Agric. 22(1 & 2): 11-18, 2011 ISSN 1017-8139

7. UC Pest Management Guidelines, available in http://ipm.ucanr.edu/PMG/r116301111.html

Smart Gardener, Plant Guide, available in https://www.smartgardener.com/plants/6865-bean-specialty-hyacinth-lablah-purpureas/pests/538-aphids

9. Richard J. Gill, Nigel E. Raine. Chronic impairment of bumblebee natural foraging behavior induced by sublethal pesticide exposure, Functional Ecology, 2014, DOI: 10.1111/1365-2435.12292.

10. Stackpoole, S. M., Shoda, M. E., Medalie, L, Stone, W. W. 2017. Pesticides in US Rivers: Regional differences in use, occurrence, and environmental toxicity, 2013 to 2017, Science of The Total Environment, Volume 787, 15 Sep. 2021, 147147 https://doi.org/10.1016/j.scitoteny.2021.147147

I claim:

1. A new and distinct variety of fruit vine of the Cucurbitaceae class, substantially as herein shown and described, characterized by intense aroma, rapid growth; tender stems, forked tendrils, heart-shaped or palmately broad green leaves, and five-petalled, long lacy fringed white unisexual day-night open flowers; oddly shaped waxy green or white skinned fruits; resistant to major fungal, bacterial, viral diseases; repellant to aphids, insects, beetles from pumpkin, squash, and bean plants if uniformly interposed among the target plants, replacement for some environmentally harmful chemical pesticides.
